(19) United States
(12) Patent Application Publication
(10) Pub. No.: US 2022/0249371 A1
(43) Pub. Date: Aug. 11, 2022

(54) Extended Release Vitamin C and Manufacturing Thereof

(71) Applicant: Capsugel Belgium NV, Bomem (BE)

(72) Inventors: Tyler White, Greenwood, SC (US); Kelli Fowler, Greenwood, SC (US); Jowell Bolivar, Greenwood, SC (US)

(21) Appl. No.: 17/665,680

(22) Filed: Feb. 7, 2022

Related U.S. Application Data
(60) Provisional application No. 63/146,863, filed on Feb. 8, 2021.

(51) Int. Cl. A61K 9/107; A61K 31/375; A61K 47/54

(52) U.S. Cl. CPC A61K 9/1075; A61K 47/544; A61K 31/375

(57) ABSTRACT

The present disclosure is directed to an extended release composition containing Vitamin C in a lipid matrix, which releases the Vitamin C over a period of time.

### CROSS REFERENCE TO RELATED APPLICATIONS

This application claims priority to and the benefit of U.S. Provisional Application No. 63/146,863, filed Feb. 8, 2021, the contents of which are incorporated herein by reference.

### BACKGROUND

Vitamin C as L-ascorbic acid, is a water-soluble vitamin that is naturally present in some foods, added to others, and available as a dietary supplement. Unlike most animals, humans are unable to synthesize vitamin C endogenously, hence Vitamin C is added to diets, thru food, drinks or supplements. Moreover, Vitamin C vital for the biosynthesis of collagen, L-carnitine, and certain neurotransmitters and Vitamin C is also involved in protein metabolism. Likewise, Vitamin C plays an important role in immune function. Insufficient vitamin C intake causes scurvy, which is characterized by fatigue or lassitude, widespread connective tissue weakness, and capillary fragility.

Inside the body, absorption of Vitamin C typically takes place in the intestinal tract. However, Vitamin C can also be destroyed by stomach acid, and the amount of Vitamin C intake to elicit nutritional benefits would have to be increased. There is a need in the art to release Vitamin C in the intestines rather than the stomach, therefore eliminating the degradation of additional Vitamin C in the stomach or upper portion of the gastrointestinal tract, and allowing for increased absorption. Also it should be noted that the body has slow absorption processes for Vitamin C, and the amount that can be absorbed is limited over a short period of time. Thus, there is a further need to have a slower release of Vitamin C inside the body. The present disclosure provides a solution to these needs.

### SUMMARY

In general, the present disclosure is directed to an extended release composition containing L-ascorbic acid or derivatives thereof, the composition having a delayed release profile to allow the L-ascorbic acid or derivatives thereof to release in the intestines rather than the stomach, allowing for increased absorption and stopping the degradation of additional L-ascorbic acid or derivatives thereof administered to a mammal, in the upper portion of the gastrointestinal tract, including the stomach. In addition, the extended release of the L-ascorbic acid to the body over an extended period of time will further improve the body's ability to absorb L-ascorbic acid. In one aspect, for instance, the present disclosure is directed to a dietary supplement composition in which amounts of L-ascorbic acid or derivatives thereof are contained or dispersed within an edible lipid system that is capable of delivering effective amounts of L-ascorbic acid or derivatives thereof to a mammal for various other health benefits. In a particular aspect of the disclosure, the edible lipid system is a lipid multiparticulate. In addition, through the methods and compositions of the present disclosure, the bioavailability of L-ascorbic acid or derivatives thereof compound can be greatly enhanced in a mammal. Bioavailability can be further improved with the addition of a lecithin phospholipid in addition to the ascorbic acid or a derivative thereof.

In a further aspect of the present disclosure, the L-ascorbic acid or derivatives thereof is released from the composition over a period up to about 30 hours after ingestion by a user, such as in period of time from about 0.5 hours to 24 hours after ingestion, and more particularly in a period of time from about 1 hour to about 20 hours after ingestion.

Another feature of the present disclosure, the L-ascorbic acid or derivatives thereof is encapsulated by the lipid matrix. Further, the active agent is present in the lipid multiparticulate particles in an amount from about 1% to about 80% by weight, such as in an amount from about 10% to about 75% by weight, more particularly in an amount from about 25% to about 70% by weight based on the total weight of the lipid multiparticulate particles. The lipid multiparticulate particles have an average particle size of greater than 1 μm, generally greater than 10 μm, typically from about 40 microns to about 3000 microns, such as from 100 microns to 2000 microns.

In one particular aspect of the present disclosure, the lipid matrix contains at least one low flow point excipient and at least one high flow point excipient. Typically the low flow point excipients are present in the composition in an amount of from about 0.1% to about 20% by weight and wherein the high flow point excipients are present in the composition in an amount of from about 20% to about 85% by weight based on the total weight of the composition.

In a further embodiment of the present disclosure, the lipid matrix contains a fatty alcohol, a fatty acid, a fatty acid ester of a glycol and a poly glycol, a fatty acid ester of glycerol, polyglycerol, a polyglycolized glyceride, a C10-C18 triglycerides stearoyl polyoxylglyceride, a lauroyl macrogol-32 glyceride, a caprylocaproyl macrogol-8 glyceride, an oleoyl macrogol-6 glyceride, a linoleoyl macrogol-6 glyceride, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, a propylene glycol fatty acid ester, esterified alpha-tocopheryl polyethylene glycol succinate, a propylene glycol monolaurate (C12) ester, polyoxyl 35 castor oil, polyoxyl 40 hydrogenated castor oil, a lecithin, vitamin E, tocopheryl polyethylene glycol succinate (TPGS), a sugar fatty acid ester, a sorbitan fatty acid ester, a polyoxyethylene sorbitan fatty acid ester, a polyoxyethylene-polyoxypropylene copolymer, rosemary extract, propylene glycol, triacetin, isorpropyl myristate, diethylene glycol monoethyl ether, polyethylene glycol, glycerol, mixtures or combinations thereof.

In another embodiment of the present disclosure, the lipid matrix contains a wax, a fatty alcohol, and a fatty acid. In a particular embodiment, the wax comprises candelilla wax, wherein the fatty alcohol comprises stearyl alcohol, and wherein the fatty acid comprises stearic acid.

In an further embodiment, the lipid matrix may further contain a surfactant. Suitable surfactants include, for example, polysorbate, a laureth sulfate, or mixtures thereof.

The lipid matrix may further contain other additional ingredients, such as flow aids, antioxidant, dispersing agent and/or a flavoring or sweetener.

In an aspect of the present disclosure, the extended release composition particles, may be placed into a capsule, formed into a tablet, placed in a softgel, placed in a gummy, may be alternatively ingested directly by a mammal as a powder or can be incorporated into a beverage or other food item.

In another embodiment of the present disclosure, provided is a method for administering L-ascorbic acid or derivatives thereof to a mammal over an extended period of time. The method comprising orally administering to a mammal an extended release composition having lipid multiparticulate particles, the lipid multiparticulate particles containing a lipid matrix. The active agent is dispersed in the lipid matrix and wherein the active agent comprising a L-ascorbic acid or derivatives thereof. Each dosage administered to the mammal containing the L-ascorbic acid or derivatives thereof in an amount from about 1 mg to about 2,000 mg, for example, 2 mg to about 1000 mg and more particularly between about 5 mg to 500 mg.

In a yet a further embodiment provided is a method of increasing the bioavailability of L-ascorbic acid or derivatives thereof in a mammal, and said method comprises forming an extended release composition described above in any one of the previous aspects and embodiments of the present disclosure and administering the extended release composition to the mammal.

In another embodiment of the present disclosure, provided is a nutraceutical composition containing the extended release composition described above in any one of the previous aspects and embodiments of the present disclosure and a second nutraceutical ingredient. One example of a second nutraceutical ingredient comprises undenatured collagen.

Other features and aspects of the present disclosure are discussed in greater detail below.

### BRIEF DESCRIPTION OF DRAWINGS

FIG. 1 graphically shows the release date from Example 5.

### DEFINITIONS

As used in this application and in the claims, the singular forms “a,” “an,” and “the” include the plural forms unless the context clearly dictates otherwise. Additionally, the term “includes” means “comprises.” The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

Unless otherwise indicated, all numbers expressing quantities of ingredients, properties such as molecular weight, percentages, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited. As used herein, the terms “about,” “approximately,” or “generally,” when used to modify a value, indicates that the value can be raised or lowered by 10% and remain within the disclosed aspect.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” means by weight as a percentage of the total weight or relative to another component in the composition.

The phrase “effective amount” means an amount of a compound that promotes, improves, stimulates, or encourages a response to the particular condition or disorder or the particular symptom of the condition or disorder.

The term “therapeutically effective amount” as used herein, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “nutraceutical” and refers to any compound added to a dietary source (e.g., a food, beverage, or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved joint health, resilience, and recovery, and can include without limitation human, canine, equine, feline, bovine, ovine, or porcine mammals. For purposes of this application, “mammal” does include human subjects.

The term “supplement” means a product in addition to the normal diet but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

As used herein, “healthy” refers to the absence of illness or injury.

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

The present disclosure is generally directed to lipid multiparticulates containing L-ascorbic acid or derivatives thereof. The particles, may be placed into a capsule, formed into a tablet, placed in a softgel, placed in a gummy, may be alternatively ingested directly by a mammal as a powder or can be incorporated into a beverage or other food item. The lipid multiparticulate particles include a lipid matrix that, in one embodiment, can be formulated to release the L-ascorbic acid or derivatives thereof when the particles are in contact within an environment which cause the L-ascorbic acid or derivatives thereof to be released from the lipid multiparticulates, such as in the digestive systems of a mammal that has been orally administered or otherwise ingested the lipid multiparticulates.

The following description is exemplary in nature and is not intended to limit the scope, applicability or configuration of the invention in any way. Various changes to the described embodiments may be made in the function and arrangement of the elements described herein without departing from the scope of the disclosure.

As to further definitions in the present disclosure are as follows:

As used herein, the term “Vitamin C” means ascorbic acid and its derivatives. Such derivatives include, for example, oxidation products such as dehydroascorbic acid and edible salts of ascorbic acid such as, illustratively, calcium, sodium, magnesium, potassium and zinc ascorbates. The term vitamin C includes these derivatives and any other art-recognized ascorbic acid derivatives including ascorbic acid esters, for example ascorbyl palm itate and ascorbyl stearate. In a particular embodiment of the present disclosure, Vitamin C is L-ascorbic acid.

As used herein, the term “flow point” is the temperature at which any portion of the mixture becomes sufficiently fluid that the mixture, as a whole, may be atomized. Generally, a mixture is sufficiently fluid for atomization when the viscosity of the molten mixture is less than 20,000 cp, or less than 15,000 cp, or less than 10,000 cp, less than 5000 cp, or even less than 1000 cp. The viscosity can be measured by a controlled stress rheometer, which measures viscosity as a function of temperature, and may use either a shear-type or rotational rheometer. As used herein, melting point refers to the temperature that marks the midpoint of the transition from a solid crystalline or semi-crystalline state to a liquid state. As measured by DSC and other melting point apparatuses, the melting point is the temperature where upon heating the solid material, the maximum exothermic heat flow occurs. In general, melting point will be used in reference to relative pure single component materials such as some actives or essentially single component excipients (e.g. stearyl alcohol) and flow point will be used in reference to multi-component materials or mixtures.

As used herein, the term “semi-solid” is a solid at ambient temperature (23° C.) but becomes a liquid at temperatures above 30° C. or 40° C., or at body temperature.

Unless otherwise indicated, “capsule” means a container suitable for enclosing solids or liquids and includes empty capsule shells and components thereof such as caps and bodies that may be assembled together to form the capsule.

Unless otherwise indicated, “dosage form” refers to a solid composition comprising an active ingredient. The active ingredient or contents of the dosage form may be solid, semi solid or liquid.

As used herein, the term “particle” refers a portion or quantity of material(s), such as a small portion or quantity of material(s). For example, as provided herein, the term particle may refer generally to a composition containing a core and one or more outer layers surrounding the core. In some embodiments, the particle(s) described may be generally spherical in shape. The term “particle” as used herein includes or may be used interchangeably with the following: pellet, beadlet, multiparticulates, particulates, spheres, including microspheres, seeds, and the like. The term particle as used herein is not limited to only a particle formed by certain methods or processes. Indeed, the particle(s) described herein may be formed by any suitable process. Certain suitable processes include, but are not limited to, melt spray congealing, spheronization, extrusion, compression, powder layering, liquid layering, pelletization by melt and wet granulation, and combinations thereof. The particle(s) as described herein may be solid or semi-solid particles. In some embodiments, the particles describe herein can include both solid and semi-solid compositions contained on or within the particle itself.

Embodiments of the disclosed composition may include at least one active ingredient or active agent. The compositions may contain one or more active ingredients. As used herein, by “active” or “active ingredient” is meant a drug, medicament, pharmaceutical, therapeutic agent, nutraceutical, or other compound that may be desired to be administered to the body. The active ingredient may be a “small molecule,” generally having a molecular weight of 2000 Daltons or less. The active ingredient may also be a “biological active.” Biological active ingredients include proteins, antibodies, antibody fragments, peptides, oligonucleotides, vaccines, and various derivatives of such materials. In one embodiment, the active ingredient is a small molecule. In another embodiment, the active ingredient is a biological active. In still another embodiment, the active ingredient is a mixture of a small molecule and a biological active. Also as used herein, the terms “active ingredient”, “first active ingredient”, “second active ingredient”, etc. may be used to denote active ingredients located in different places within the particle, such as those located in the core or those located in the one or more outer layers. However, the terms “first” or “second” do not necessarily denote that the first active ingredient is different from the second active ingredient. For example, in certain embodiments, the active ingredient contained within the core may be the same as the second active ingredient contained within an outer layer disposed on the core. While in certain other embodiments, the active ingredient contained within the core may be different from the second active ingredient contained within an outer layer disposed on the core.

As described above, in one embodiment, the active ingredient can be L-ascorbic acid or derivative thereof which are incorporated or dispersed into a lipid matrix. In one embodiment, the composition of the present disclosure extended release composition comprising a lipid multiparticulate that delays the release of L-ascorbic acid or derivative thereof beyond the initial time when the extended release composition enters the digestive system of a mammal, such as a human, to deliver a fairly constant dose of the L-ascorbic acid or derivative thereof to the mammal over a period of time. For example, the L-ascorbic acid or derivatives thereof can be dispersed or encapsulated within a lipid matrix that is specially formulated to entrap the L-ascorbic acid or derivative thereof and postpone their release from the lipid matrix for a period of time. Of particular advantage, the particles of the present disclosure can be constructed to be 100% vegetarian. In addition, the particle size can be carefully controlled and adjusted to fit different purposes, such as when producing capsules, beverages, tablets, and the like.

In one embodiment, to increase the bioavailability of the ascorbic acid or derivative thereof, it has been discovered that the addition of lecithin/phospholipid to the formulation with in the lipid multiparticulate composition, the bioavailability of the ascorbic acid or derivative there or can be increase. Suitable lecithin/phospholipid compound compounds such as sunflower lecithin, which have a high content of phosphatidylcholine. The lecithin/phospholipid can be present in any amount, however, the higher the content in the lipid multiparticulates, the faster the ascorbic acid or derivative is released from the multiparticulates. Generally the lecithin/phospholipid is added in an amount up to about 25% of the weight of the multiparticulate. Generally, it is used in amounts of less than 15% by weight.

Lipid products made in accordance with the present disclosure, however, can be made very economically and can contain amounts L-ascorbic acid or derivatives thereof. The composition of the present disclosure, for instance, can contain L-ascorbic acid or derivatives thereof, in an amount greater than about 1% by weight, such as in an amount greater than about 5% by weight, such as in an amount greater than about 10% by weight, such as in an amount greater than about 15% by weight, such as in an amount greater than about 20% by weight, such as in an amount greater than about 25% by weight, such as in an amount greater than about 30% by weight. L-ascorbic acid or derivatives thereof can be present in the composition in an amount less than about 80% by weight, such as in an amount less than about 75% by weight, such as in an amount less than about 70% by weight, based on the total weight of the lipid multiparticulate particles containing L-ascorbic acid or derivatives thereof.

The lipid matrix used to form the particles of the present disclosure, for instance, can be made from or can include many different lipid-based components, various different acid-resistant components, and the like. Examples of materials that can be used to form the liquid matrix include a fatty alcohol, a fatty acid, a fatty acid ester of a glycol and a poly glycol, a fatty acid ester of glycerol, polyglycerol, a polyglycolized glyceride, a C10-C18 triglycerides stearoyl polyoxylglyceride, a lauroyl macrogol-32 glyceride, a caprylocaproyl macrogol-8 glyceride, an oleoyl macrogol-6 glyceride, a linoleoyl macrogol-6 glyceride, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, a propylene glycol fatty acid ester, esterified alpha-tocopheryl polyethylene glycol succinate, a propylene glycol monolaurate (C12) ester, polyoxyl 35 castor oil, polyoxyl 40 hydrogenated castor oil, a lecithin, vitamin E, tocopheryl polyethylene glycol succinate (TPGS), a sugar fatty acid ester, a sorbitan fatty acid ester, a polyoxyethylene sorbitan fatty acid ester, a polyoxyethylene-polyoxypropylene copolymer, propylene glycol, triacetin, isorpropyl myristate, diethylene glycol monoethyl ether, polyethylene glycol, glycerol, mixtures or combinations thereof.

In one embodiment, the liquid matrix is formed from at least one low flow point excipient and at least one high flow point excipient.

For example, in certain embodiments the lipid matrix may contain one or more low-flow point excipients. Low flow point excipients generally include fatty alcohols, fatty acids, fatty acid esters of glycols and poly glycols, fatty acid esters of polyglycerol and fatty acid esters of glycerol (glycerides) with flow points of less than 50° C. When the low flow point excipient is a relatively pure material, the melting point is also less than 50° C. A preferred class of low flow point excipients are low flow point glycerides. By “low flow point” excipient, such as a glyceride, is meant that the melting point of the excipient, such as a glyceride, is less than 50° C. In some embodiments, the low flow point glyceride has a melting point of less than 40° C. In some embodiments, the low-flow point excipient, such as glyceride, is a mixture of compounds, having a flow point of 50° C. or less. In some embodiments, the low-flow point excipient, such as glyceride, has a flow point of 40° C. or less. In some embodiments, the low-flow point glyceride has a low flow point of 30° C. or less. Exemplary low flow point glycerides include polyglycolized glycerides, such as some of the Gelucire products manufactured by Gattefosse, such as Gelucire® 43/01 having a nominal melting point of 43° C. Mixtures of low flow point glycerides are also effective, such as mixtures of Gelucire® 43/01 (C10-C18 triglycerides), Gelucire® 50/13 (stearoyl polyoxylglycerides), Gelucire® 44/14 (lauroyl macrogol-32 glycerides), and mixtures thereof. Other glycerides may also be used, such as fatty acid esters of glycols and poly glycols, and fatty acid esters of polyglycerols.

A function of the low flow point excipient is to ensure that at least a significant portion of the formulation matrix softens when ingested orally by a patient, at the temperature of the GI tract (about 37° C. for humans). This allows the formulation to break down by digestion in the gastro-intestinal (GI) tract, and ultimately to disperse in the GI tract to promote dissolution and absorption of the active. In certain embodiments the low flow point excipient provides a significant portion of the formulation matrix to be present in a non-crystalline liquid or amorphous state when ingested and softened in the GI tract.

Exemplary low flow point fatty alcohols include myristyl alcohol (Tm 38° C). , lauryl alcohol (Tm 23° C). and capric alcohol (Tm 7° C).

Exemplary low flow point fatty acids include lauric acid (Tm 44° C). and oleic acid (Tm 16° C).

In certain embodiments, the lipid matrix includes a high-flow point excipient. For example, in certain embodiments the lipid matrix may contain one or more high-flow point excipients. By “high flow point” excipient is meant an excipient that has a flow point 50° C. or more. High flow point excipients may also have a melting point above 50° C. High flow point excipients generally include fatty alcohols, fatty acids, fatty acid esters of glycols and poly glycols, fatty acid esters of polyglycerol, fatty acid esters of glycerol (glycerides), waxes, polar waxes and other materials with flow points of greater than 50. A preferred class of high flow point excipients are “high flow point glycerides”. By high flow point glyceride is meant that the flow point or melting point of the glyceride is 50° C. or more. In some embodiments, the high flow point glyceride has a melting point of 60° C. or more. In some embodiments, the high-melting point glyceride is a mixture of compounds, having a flow point of 50° C. or more. In some embodiments, the high-flow point glyceride has a flow point of 60° C. or more. In some embodiments, the high flow point glyceride has a flow point of 70° C. or more.

Exemplary high flow point glycerides include glycerol behenate, glycerol dibehenate, glycerol palmitate, hydrogenated castor oil, and mixtures thereof.

Often, the high flow point glyceride is a mixture of compounds that are formulated into a product and sold under a variety of trade names.

Exemplary high flow point and high melt point fatty alcohols include stearyl alcohol (Tm 58° C). and behenyl alcohol (Tm 71° C).

Exemplary high flow point and high melt point fatty acids include palm itic acid (Tm 63° C). and stearic acid (Tm>70° C).

Exemplary waxes include paraffin wax, beeswax, candelilla wax, carnauba wax, and mixtures thereof.

A function of the high flow point excipient is to aid in the manufacturability of the particles by enabling the particles to congeal at a lower temperature to obtain solid particles during the melt-spray-congeal processing. In certain embodiments the high flow point excipient aids the physical stability of the formulation. In most embodiments, the high flow point excipient is not appreciably digested in the GI tract.

In some embodiments, the lipid matrix of the particles may include other excipients to improve the performance and chemical stability of the formulations. In some embodiments, a dispersing agent is included in the particles. Exemplary dispersing agents include lecithin, glycerol monostearate, ethylene glycol palm itostearate, aluminum oxide, polyethylene alky ethers, sorbitan esters, and mixtures thereof. In one embodiment, the particles include an antioxidant to maintain chemical stability of the active agent. Exemplary antioxidants include vitamin E, tocopheryl polyethylene glycol succinate (TPGS), rosemary extract, ascorbic acid, asorbyl palmitate, butylated hydroxyanisole (BHA), buytlated hydroxytoluene (BHT), and mixtures and combinations thereof.

In some embodiments, a flow aid is used to improve the flow properties of the particles. Exemplary flow aids also known as glidants include silica, calcium silicate, cab-o-sil, silicon dioxide, calcium phosphate tribasic, colloidal silicon-dioxide, magnesium silicate, magnesium trisilicate, starch, talc, and other flow aids.

In one aspect, the dietary composition further contains a disintegrating agent. The disintegrating agent, for example, can be a cross-linked carboxymethyl cellulose, such as croscarmellose. Croscarmellose is a cross-linked carboxymethyl cellulose salt. In one aspect, the cross-linked carboxymethyl cellulose can be a sodium salt. In one embodiment, the cross-linked carboxymethyl cellulose can be in the form of fibers or particles. The fibers or particles can form a free—flowing powder that is typically white in color. The cross-linked carboxymethyl cellulose is hydrophilic but also insoluble. Once placed in contact with a liquid, the cross-linked carboxymethyl cellulose wicks the fluid and begins to swell. The swelling action of the cross-linked carboxymethyl cellulose causes the dietary composition to disintegrate. In this manner, the cross-linked carboxymethyl cellulose can be used to control the release of the L-ascorbic acid or derivatives thereof.

The ability of the disintegrating agent to affect release of the L-ascorbic acid or derivatives thereof can be controlled by controlling the type of cross-linked carboxymethyl cellulose incorporated into the composition and by controlling the amount of the disintegrating agent added to the composition. For example, the ability of the cross-linked carboxymethyl cellulose to swell can depend upon the hydration of the carboxymethyl groups by controlling the degree of substitution within the cross-linked cellulose polymer. The degree of substitution, for instance, can be greater than about 0.5, such as greater than about 0.55, such as greater than about 0.6, such as greater than about 0.65, such as greater than about 0.7, such as greater than about 0.75, such as greater than about 0.8. The degree of substitution is generally less than about 0.9, such as less than about 0.85, such as less than about 0.8, such as less than about 0.75. The degree of substitution can be determined by elemental analysis.

The amount of the disintegrating agent or the cross-linked carboxymethyl cellulose incorporated into the dietary composition can generally be greater than about 0.5% by weight, such as greater than about 1% by weight, such as greater than about 3% by weight, such as greater than about 5% by weight, And generally less than about 15% by weight, such as less than about 12% by weight, such as less than about 10% by weight, such as less than about 8% by weight.

The particles described herein are solid at ambient temperature and are generally spherical in shape. By generally spherical is meant that while most particles are essentially spherical, they do not necessarily form “perfect” spheres. Such particle variations in spherical shapes are known to those persons of ordinary skill in the art of melt-spray-congeal processing and similar particulate forming methods.

The particles may have a size ranging from an average diameter greater than about 1 μm, and generally greater that about 10 μm. Typically the particles have a size ranging from an average diameter about 40 μm to about 3000 μm, such as from about 50 μm to about 2500 μm, such as from about 80 μm to about 2000 μm, such as from about 100 μm to about 1500 μm, such as from about 200 μm to about 1000 μm, such as from about 300 μm to about 800 μm. To measure the diameters of the particulates, there are several methods that can be used, including laser diffraction, optical microscopy, and/or SEM.

In certain embodiments, the particles containing the active ingredient and lipid matrix have a flow point above 25° C., such as above 30° C., such as above 35° C., such as above 40° C.

In one embodiment, the lipid matrix composition comprises greater than 50 wt % of the low flow point excipient. In one embodiment, the lipid matrix composition comprises at least 2 wt % of the high flow point excipient. In another embodiment, the lipid matrix composition comprises less than 20 wt % of the high flow point excipient. In another embodiment the mass ratio of the low flow excipient to the high flow excipient is at least 2:1. In still another embodiment, the mass ratio of the low flow excipient to the high flow excipient is at least 3:1. In another embodiment, the mass ratio of the low flow excipient to the high flow excipient is at least 4:1. In another embodiment, the mass ratio of the low flow excipient to the high flow excipient is at least 10:1. In another embodiment, the mass ratio of the low flow excipient to the high flow excipient is at least 15:1. In another embodiment, the mass ratio of the low flow excipient to the high flow excipient is at least 20:1.

In another aspect, the lipid matrix composition contains greater than 50% by weight of one or more high flow point excipients. For example, in one embodiment, the lipid matrix is made exclusively from one or more high flow point excipients and does not contain a low flow point excipient. One or more high flow point excipients, for instance, can be present in the lipid matrix in an amount greater than about 40% by weight, such as an amount greater than about 50% by weight, such as in an amount greater than about 60% by weight, such as in an amount greater than about 65% by weight, such as in an amount greater than 70% by weight, and generally in an amount less than about 98% by weight, such as in an amount less than about 95% by weight, such as in an amount less than about 90% by weight, such as in an amount less than about 80% by weight, such as in an amount less than about 70% by weight. When greater amounts of high flow point excipients are present, one or more low flow point excipients may be present in the composition in an amount less than about 30% by weight, such as in an amount less than about 20% by weight, such as in an amount less than about 10% by weight and generally in an amount greater than 25% by weight, such as in an amount greater than about 4% by weight. The mass ratio of the high flow point excipients to the low flow point excipients can be from about 100:1 to about 1:1, such as from about 50:1 to about 10:1, such as from about 20:1 to about 5:1.

In one particular embodiment, the lipid matrix contains a wax combined with a fatty acid alcohol and a fatty acid. The wax, for instance, can comprise candelilla wax. The fatty alcohol, on the other hand, can be stearyl alcohol, while the fatty acid can be stearic acid. For example, the wax, such as candelilla wax, can be present in the composition in an amount greater than about 20% by weight, such as in an amount greater than about 25% by weight, and generally in an amount less than about 50% by weight, such as in an amount less than about 45% by weight. The fatty alcohol, on the other hand, can generally be present in an amount greater than about 10% by weight, such as in an amount greater than about 12% by weight, and generally in an amount less than about 25% by weight, such as in an amount less than about 22% by weight, such as in an amount less than about 18% by weight. The fatty acid, on the other hand, can be present in the composition in an amount greater than about 3% by weight, such as in an amount greater than about 5% by weight, such as in an amount greater than 7% by weight, and generally in an amount less than about 15% by weight, such as in an amount less than about 12% by weight, such as in an amount less than about 10% by weight.

The lipid matrix may also comprise a dispersing agent. In one embodiment, the lipid matrix is comprised of from 0 wt % to 20 wt %, such as from 0.01 wt % to 20 wt %, of a dispersing agent. In another embodiment, the lipid matrix is comprised of from 2 wt % to 10 wt % of a dispersing agent.

The lipid matrix may also comprise an antioxidant. In one embodiment, the lipid matrix comprise from 0 wt % to 20 wt %, such as from 0.01 wt % to 10 wt %, of an antioxidant. In one embodiment, the lipid matrix comprise from 1 wt % to 5 wt % of an antioxidant.

The lipid matrix may also comprise a flow aid. In one embodiment, the lipid matrix may comprise from 0 wt % to 5 wt %, such as from 0.01 wt % to 5 wt %, of a flow aid. In another embodiment, the lipid matrix may comprise from 0.5 wt % to 2 wt % of a flow aid.

The lipid matrix may also contain flavoring or sweeteners to improve the taste of the particles to the user. In one embodiment, the lipid matrix comprise from 0 wt % to 15 wt %, such as from 0.01 wt % to 10 wt %, of an flavoring or sweetener. In one embodiment, the lipid matrix comprise from 1 wt % to 5 wt % of an antioxidant flavoring or sweetener. Flavoring and sweeteners include essential oils other sweeteners used in the nutraceutical or food industries.

The lipid matrix described herein may be formulated by any suitable process. In some embodiments, the matrix may be formulated by a suitable melt-spray-congeal process.

A molten mixture is formed by mixing and heating the lipid matrix compositions as previously described. “Molten mixture” means that the mixture of an active ingredient and lipid matrix materials are sufficiently mixed and heated to fluidize the mixture sufficiently to allow it to be atomized into droplets. Generally, the mixture is molten in the sense that it will flow when subjected to one or more forces such as pressure, shear, and centrifugal force, such as that exerted by a centrifugal or spinning-disk atomizer.

Once the molten mixture has been formed, it is delivered to an atomizer that breaks the molten mixture into small droplets. Virtually any method can be used to deliver the molten mixture to the atomizer. In certain embodiments of the disclosed methods the molten mixture is delivered to the atomizer by use of pumps and/or various types of pneumatic devices such as pressurized vessels or piston pots or extruder. In certain embodiments the molten mixture is maintained at an elevated temperature during delivery to the atomizer to prevent its solidification and to keep it in a flowable state.

When a centrifugal atomizer (also known as rotary atomizers or spinning-disk atomizer) is used, the molten mixture is fed onto a rotating surface, where it spreads outward and flows by centrifugal force. The rotating surface may take several forms, examples of which include a flat disk, a cup, a vanned disk, and a slotted wheel. The surface of the disk may also be heated to aid in atomization of the molten mixture or cooled to aid in the solidification of the cores containing the lipid matrix. Several mechanisms of atomization are observed with flat-disk and cup centrifugal atomizers, depending on the flow of molten mixture to the disk, the rotation speed of the disk, the diameter of the disk, the viscosity of the feed, and the surface tension and density of the feed. At low flow rates, the molten mixture spreads out across the surface of the disk and when it reaches the edge of the disk, forms a discrete droplet, which is then flung from the disk.

Once the molten mixture has been atomized, the droplets are congealed, typically by contact with a gas at a temperature below the solidification temperature of the composition. Typically, it is desirable that the droplets are congealed in less than 60 seconds, less than 10 seconds, or even in less than 1 second. In certain embodiments congealing at ambient temperature using an ambient temperature cooling medium, results in sufficiently rapid solidification of the droplets. However, as certain embodiments of the disclosed compositions are comprised of at least 50 wt % of a low flow point excipient, it is often preferred to utilize a cooling medium that is at a temperature that is at least 10° C. below ambient temperature. For some embodiments, it is preferred to utilize a cooling medium that is at least 20° C., below ambient temperature.

In one aspect, one or more surfactants can optionally be incorporated into the composition. Surfactants can be incorporated into the composition for various reasons. It was discovered that some surfactants can actually facilitate control of the delayed release function of the composition. In some embodiments, surfactants and co-surfactants may be included in the compositions. Exemplary surfactants and co-surfactants include polyethoxylated 12-hydroxysteric acid, also known as PEG15 hydroxystearate (Kolliphor® HS-15), propylene glycol monocaprylate (C8) esters (Caproyl™ 90), esterified alpha-tocopheryl polyethylene glycol succinate (TPGS), mono, di, tricaprylic (C8) and capric acid (C10) esters of glycerol and mono and diesters of PEG400 (Labrasol®), Propylene glycol monolaurate (C12) esters (Labrafil® M1944CS), Polyoxyl 40 hydrogenated castor oil (Kolliphor® RH40), lecithins, and mixtures thereof.

In one embodiment, the surfactant incorporated into the composition can be a polysorbate, a sulfate surfactant, or mixtures thereof. Sulfate surfactants include, for instance, salts of fatty acids sulfates. For example, in one embodiment, the surfactant can be sodium laureth sulfate.

The amounts of surfactants incorporated into the composition can vary widely depending upon the reason for adding the surfactant or the desired result. In general, when included in the composition, one or more surfactants can be present in an amount greater than about 1% by weight, such as in an amount greater than about 3% by weight, such as in an amount greater than about 7% by weight, such as in an amount greater than about 10% by weight, such as in an amount greater than about 15% by weight, such as in an amount greater than about 20% by weight, such as in an amount greater than about 25% by weight, such as in an amount greater than about 30% by weight. One or more surfactants are generally present in the composition in an amount less than about 50% by weight, such as in an amount less than about 40% by weight, such as in an amount less than about 30% by weight, such as in an amount less than about 20% by weight, such as in an amount less than about 10% by weight.

Other benefits of the lipid multiparticulates is that the L-ascorbic acid or derivatives thereof can be in products such as nutritional bars; and in sachet formats for adding in to oatmeal, cereals, ready-to-mix (RTM) type beverages, salads, and other similar food products to achieve the benefits of the L-ascorbic acid or derivatives thereof.

In some embodiments, the one or more particles provided herein may be formulated into any suitable dosage formulation. For example, in certain embodiments, the one or more particles provided herein may be placed into a capsule for delivery by oral ingestion. Exemplary capsules include hard gelatin capsules, soft gelatin capsules, HPMC capsules, as well as capsules made from other materials. The one or more particles may be suspended in an aqueous-based matrix or an oil-based matrix within the capsule itself. In certain embodiments where the particles are suspended in an aqueous-based matrix or an oil-based matrix, the aqueous-based matrix or oil-based matrix may additionally include one or more active ingredients. In certain embodiments, the one or more particles may be contained within a monolithic enteric capsule suitable for providing a modified release profile when ingested.

Capsules normally include a shell filled with one or more specific substances. The shell itself may be a soft or a hard capsule shell. Hard capsule shells are generally manufactured using dip molding processes, which can be distinguished into two alternative procedures. In the first procedure, capsules are prepared by dipping stainless-steel mold pins into a solution of polymer, optionally containing one or more gelling agents (e.g. carrageenans) and co-gelling agents (e.g. inorganic cations). The mold pins are subsequently removed, inverted, and dried to form a film on the surface. The dried capsule films are then removed from the molds, cut to the desired length, and then the telescoping fit caps and bodies are assembled together, printed, and packaged. In the second procedure, no gelling agents or co-gelling agents are used and film-forming polymer solution gelification on the molding pins is thermally induced by dipping pre-heated molding pins into the polymer solution. This second process is commonly referred to as thermogellation, or thermogelling dip molding. The aforementioned manufacturing processes involve the use of solutions of the different ingredients that are needed for the making the telescoping fit hard capsule shells.

Hard capsules may be filled with active ingredients, such as the particles described herein, via procedures known in the art. Typically, active ingredients are combined with various compatible excipients for ease of fill. The resulting fill may be a dry powder, a granulation, particles, lipid particles, a suspension, or a liquid. Additionally, stable, filled hard capsules have advantages over other dosage delivery forms such as liquids and solid tablets. Certain active ingredients may be difficult to formulate into dry granules or may be otherwise incompatible with the tableting process. Another consideration is improved patient compliance for taste-masking and ease of swallowing, i.e., capsules being preferred by consumers over tablets. For example, in some embodiments, provided is a pharmaceutical composition that contains a capsule filled with the one or more particles disclosed herein. In some embodiments, the one or more particles have not been enterically coated for modified release or gastric protection.

In certain other embodiments, the one or more particles can be administered orally as a solid, liquid, suspension, or other suitable delivery means.

The composition of particles may be administered via buccal or sublingual administration. In one embodiment, the one or more particles may be administered as a capsule, tablet, caplet, pill, troche, drop, lozenge, powder, granule, syrup, tea, drink, thin film, seed, paste, herb, botanical, and the like.

In a further embodiment of the present disclosure, the lipid multiparticulate particles described herein can be combined with or used with other nutraceutical components to form a nutraceutical composition. The lipid multiparticulates of L-ascorbic acid or derivatives thereof can be blended with other nutraceutical components which result in stable combinations of lipid multiparticulates of L-ascorbic acid or derivatives thereof and other nutraceutical ingredients in both nutraceutical finished solid and liquid dosages, as well as in food and beverage applications. Exemplary nutraceuticals which can be blended with include the collagen, including hydrolyzed collagen or undenatured collagen, including but not limited to UC-II® product available from Lonza, probiotics, for example, but not limited to TWK10® product available from Lonza, enzymes, endogenous fatty acid amides, cetylated fatty acid esters, omega-3 fatty acids, hyaluronic acids, curcuminoids, herbal and botanical extracts, carotenoids, methylsulfonylmethane (MSM), carnitine, including but not limited to, Carnipure® available from Lonza, and antioxidants, for example, Oceanix™ available from Lonza. Other nutraceutical ingredients having anti-inflammatory benefits such as turmeric curcuminoids, eggshell membrane, green lipped mussel, omegas-3 EPA and DHA, krill oil, french maritime pine bark extract (Pycnogenol®), Scutellaria baicalensis and Acacia catechu extracts (Univestin®), ashwagandha extract, rose hip extract, tart cherry extract, astaxanthin, hops extract (Perluxan®), glucosamine, chondroitin, hyaluronic acid, salmon nasal cartilage, avocado soy unsaponifiable, methylsulfonylmethane (MSM), willow bark extract, tamarind seed extract, lactobacillus and bifidobacteria probiotic strains (e.g. TWK10® product available from Lonza), palmitoylethanolamide (PEA), and cetyl myristoleate (CM), which may further eliciting anti-inflammation health benefits.

In the present disclosure, also provided is method for administering L-ascorbic acid or derivatives thereof compound to a mammal over an extended period of time. The method includes orally administering to a mammal an extended release composition comprising lipid multiparticulate particles, the lipid multiparticulate particles comprising a lipid matrix and wherein dispersed in the lipid matrix is an active agent, the active agent comprising L-ascorbic acid or derivatives thereof. In a typical dosage, L-ascorbic acid or derivatives thereof is typically administered to the mammal containing in an amount from about 1 mg to about 2,000 mg, for example, 2 mg to about 1000 mg and more particularly between about 5 mg to 500 mg. Depending on the percentage of the L-ascorbic acid or derivatives thereof in the lipid multiparticulate, the amount of the lipid multiparticulate is adjusted to achieve the correct dosage.

Nonetheless, certain embodiments of the present disclosure may be better understood according to the following examples, which are intended to be non-limiting and exemplary in nature.

### EXAMPLE 1

Two formulations were prepared from L-ascorbic acid, candelilla wax and stearic acid. The first formulation contained 63% by weight of ascorbic acid, 7% by weight candelilla wax and 30% by weight stearic acid. The second formulation contained 65% by weight of ascorbic acid, 25% by weight candelilla wax and 10% by weight stearic acid. Either formulation was made by mixing and agitating the ingredients, and the temperature was kept at 70-75° C. until ascorbic acid was completely suspended in candelilla wax and the stearic acid. The final mixture was the processed in a melt spray congeal process, producing microparticles of 500-700 microns. The release profile was tested.

### EXAMPLE 2

A formulation was prepared from L-ascorbic acid, sunflower lecithin containing about 90% phosphatidyl choline, candelilla wax, stearyl alcohol, and flavoring. Specifically, the formula contained 53% by weight of ascorbic acid, 10% by weight of sunflower lecithin, 22% by weight candelilla wax, 10% by weight stearyl alcohol, and ˜5% by weight orange oil. The formulations were made by mixing and agitating the ingredients, and temperature was kept at 70-75° C. until ascorbic acid and sunflower lecithin containing amounts of phosphatidyl choline, was completely suspended in candelilla wax. In this mixture stearyl alcohol and orange oil were added in, and the final resulting mixture was heated at the same temperature for a period of time until stearyl alcohol was completely melted and suspended. The final mixture was then processed in a melt spray congeal unit, wherein microparticles of 500-700 microns particles were recovered.

### EXAMPLE 3

A formulation was prepared from L-ascorbic acid, sunflower lecithin containing about 90% phosphatidyl choline, candelilla wax, stearyl alcohol, and flavoring. Specifically, the formulation has 42.5% by weight of ascorbic acid, 20% by weight of sunflower lecithin, 20% by weight candelilla wax, 12.5% by weight stearyl alcohol, and ˜5% orange oil. The formulations were made by mixing and agitating the ingredients, at 70-75° C. heating temperatures, until ascorbic acid and sunflower lecithin containing amounts of phosphatidyl choline were completely suspended in candelilla wax. The resulting mixture was added with 12.5% by weight of stearyl alcohol, and was heated at the same temperature for a period of time until stearyl alcohol was completely melted and suspended. The final mixture was added with orange oil, and finally processed in a melt spray congeal unit, producing microparticles of 500-700 microns.

### EXAMPLE 4

A formulation was prepared from 48.2% w/w L-ascorbic acid, 13.3% w/w sunflower lecithin containing about 90% phosphatidyl choline, 23.5% w/w candelilla wax, 10% w/w glycerol monostearate, and ˜5% w/w orange oil. The formulation was agitated until all ingredients were completely suspended at 70-75° C. heating temperatures. The final mixture was the processed in a melt spray congeal unit, and lipid microparticles from 500-700 microns were obtained.

### EXAMPLE 5

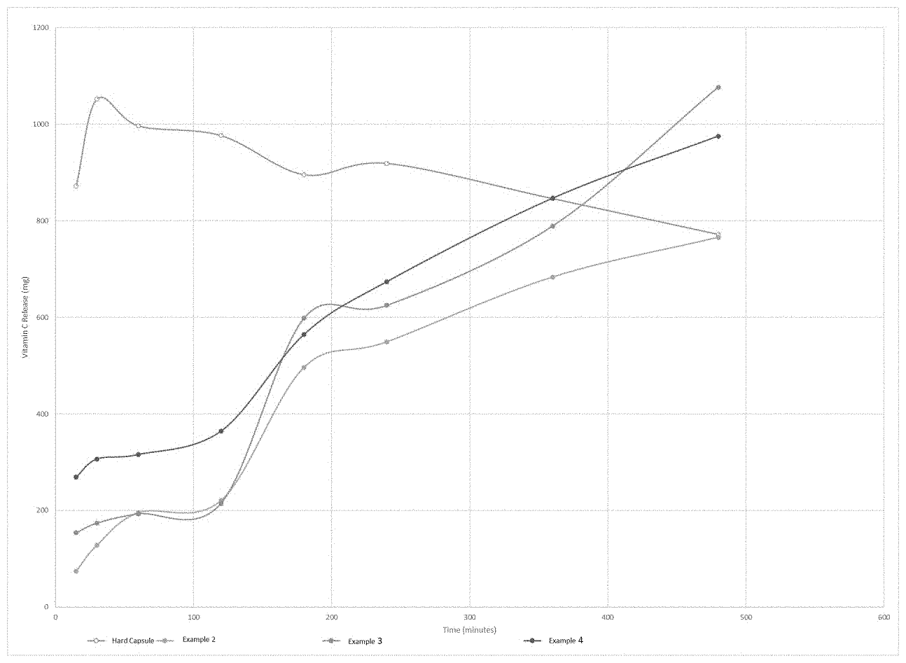

The microparticles of Examples 2, 3 and 4 were analyzed for dissolution behavior using standard and methodologies described in USP 711, Dissolution, particularly for delayed-release dosage forms. One serving of microparticles were placed in dissolution vessels with 0.1 N HCl for 120 minutes, simulating the gastric conditions, after which the dissolution media was switched to buffered 2% sodium lauryl sulfate, pH 6.8, for an additional 300 minutes (simulated intestinal digestion). Aliquots were sampled from eight (8) consecutive and different time points and were titrated for dissolved ascorbic acid concentrations, following Vitamin C ascorbic acid analyses described in several industry methodologies for Vitamin C assays. Pristine and uncoated Vitamin C in powder-filled size HPMC hard shell capsules was also subjected in the same dissolution manner for control and comparison. The results are shown in FIG. 1.

From dissolution studies, quick-release dissolution profiles for powder-filled HPMC capsule with Vitamin C ascorbic was observed. However, Vitamin C from lipid microparticles showed extended and delayed release fashion throughout the 8-hour dissolution period. The dissolution studies established that majority of protected Vitamin C from lipid multiparticulates could bypass aggressive assaults from stomach acid digestion, could be released effectively in the small intestines for better absorption.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. An extended release composition comprising:
lipid multiparticulate particles, and
an active agent, the lipid multiparticulate particles comprising a lipid matrix, and wherein the active agent is dispersed within the lipid matrix, the active agent comprising ascorbic acid or a derivative thereof, wherein the active agent is released from the lipid multiparticulate particles over a period of time.

2. The extended release composition as defined in claim 1, wherein the active agent comprises L-ascorbic acid.

3. The extended release composition as defined in claim 1, further comprising a lecithin/phospholipid.

4. The extended release composition as defined in claim 1, wherein the active agent is released from the composition over a period up to about 30 hours after ingestion by a user.

5. The extended release composition as defined in claim 1, wherein the active agent is encapsulated by the lipid matrix.

6. The extended release composition as defined in claim 1, wherein the active agent is present in the lipid multiparticulate particles in an amount from about 1% to about 80% by weight based on the total weight of the lipid multiparticulate particles.

7. The extended release composition as defined in claim 1, wherein the extended release composition is in the form of a capsule or a tablet. The extended release composition as defined in claim 1, wherein the lipid multiparticulate particles have an average particle size of from about 40 microns to about 3000 microns.

9. The extended release composition as defined in claim 1, wherein the lipid matrix comprises at least one low flow point excipient and at least one high flow point excipient.

10. The extended release composition as defined in claim 1, wherein the lipid matrix comprise a fatty alcohol, a fatty acid, a fatty acid ester of a glycol and a poly glycol, a fatty acid ester of glycerol, polyglycerol, a polyglycolized glyceride, a C10-C18 triglycerides stearoyl polyoxylglyceride, a lauroyl macrogol-32 glyceride, a caprylocaproyl macrogol-8 glyceride, an oleoyl macrogol-6 glyceride, a linoleoyl macrogol-6 glyceride, myristyl alcohol, lauryl alcohol, capric alcohol, glycerol behenate, glycerol dibehenate, glycerol palmitate, glycerol monstearate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, a propylene glycol fatty acid ester, esterified alpha-tocopheryl polyethylene glycol succinate, a propylene glycol monolaurate (C12) ester, polyoxyl 35 castor oil, polyoxyl 40 hydrogenated castor oil, a lecithin, vitamin E, tocopheryl polyethylene glycol succinate (TPGS), a sugar fatty acid ester, a sorbitan fatty acid ester, a polyoxyethylene sorbitan fatty acid ester, a polyoxyethylene-polyoxypropylene copolymer, rosemary extract, propylene glycol, triacetin, isorpropyl myristate, diethylene glycol monoethyl ether, polyethylene glycol, glycerol, mixtures or combinations thereof.

11. The extended release composition as defined in claim 1, wherein the lipid matrix comprises a wax, a fatty alcohol, and a fatty acid.

12. The extended release composition as defined in claim 11, wherein the wax comprises candelilla wax, wherein the fatty alcohol comprises stearyl alcohol, or glycerol monostearate and wherein the fatty acid comprises stearic acid.

13. The extended release composition as defined in claim 1, wherein the lipid matrix further contains a surfactant.

14. The extended release composition as defined in claim 13, wherein the surfactant comprises a polysorbate, a laureth sulfate, or mixtures thereof.

15. The extended release composition as defined in claim 9, wherein the low flow point excipients are present in the composition in an amount of from about 0.1° A to about 20% by weight and wherein the high flow point excipients are present in the composition in an amount of from about 30% to about 85% by weight based on the total weight of the composition.

16. The extended release composition as defined in claim 1, further comprising a flow aid, an antioxidant, a dispersing agent and/or a flavoring or sweetener.

17. A method for administering ascorbic acid or a derivative thereof to a mammal over an extended period of time, said method comprising:
orally administering to a mammal an extended release composition comprising lipid multiparticulate particles, the lipid multiparticulate particles comprising a lipid matrix and wherein dispersed in the lipid matrix is an active agent, the active agent comprising ascorbic acid or a derivative thereof, each dosage administered to the mammal containing the ascorbic acid or a derivative thereof in an amount from about 1 mg to about 2,000 mg.

18. A method as defined in claim 17, wherein the extended release composition is formulated such that the ascorbic acid or a derivative thereof is released from the extended release composition over a period of time from about 0.5 hours to 24 hours after administration to a user.

19. The method as defined in claim 17, wherein the extended release composition further comprises a lecithin/phospholipid.

20. A nutraceutical composition comprising the extended release composition according to claim 1 and a second nutraceutical ingredient.

21. A method of increasing the bioavailability of ascorbic acid or a derivative thereof in a mammal said method comprises forming an extended release composition according to claim 1 and administering the extended release composition to the mammal.
